(19) United States
(12) Patent Application Publication
(10) Pub. No.: US 2024/0091397 A1
(43) Pub. Date: Mar. 21, 2024

(54) OIL-LEAKAGE PREVENTION ATOMIZER AND ESSENTIAL OIL DIFFUSER

(71) Applicant: Shenzhen Parlex Household Industrial Co., Ltd., Shenzhen (CN)

(72) Inventor: SikMan TSE, Shenzhen (CN)

(21) Appl. No.: 18/512,082

(22) Filed: Nov. 17, 2023

(30) Foreign Application Priority Data
Nov. 18, 2022 (CN) 202211444044.4

(51) Int. Cl. A61L 9/14

(52) U.S. Cl. CPC A61L 9/14; A61L 2209/133; A61L 2209/134

(57) ABSTRACT

An oil-leakage prevention atomizer and an essential oil diffuser are provided. The oil-leakage prevention atomizer includes a carrier, a reaction chamber, an atomizing core, a separator, and an oil supply chamber, wherein the carrier is provided with a carrier cavity; an air inlet and an air outlet are arranged on a cavity wall of the carrier cavity; the reaction chamber is mounted in the carrier cavity and is internally provided with a reaction cavity; an oil inlet hole, an atomizing port, and an air hole are arranged on a cavity wall of the reaction cavity; the atomizing core is mounted at the atomizing port; the separator is mounted on the reaction chamber and is provided with an oil guide pipe communicated to the oil inlet hole and an air guide pipe communicated to the air hole.

### TECHNICAL FIELD

The present disclosure belongs to the technical field of atomization and essential oil diffusion equipment, and in particular, to an oil-leakage prevention atomizer and an essential oil diffuser. This application claims the priority of the preceding application No. 2022114440444, entitled: OIL-LEAKAGE PREVENTION ATOMIZER AND ESSENTIAL OIL DIFFUSER and having the priority date: Nov. 18, 2022.

### BACKGROUND

An essential oil diffuser is an aroma diffuser. A traditional essential oil diffuser is mainly composed of an atomizing component and an enclosure internally provided with an oil storage cavity. The atomizing component is mounted in the oil storage cavity and can break down an essential oil mixture in the oil storage cavity, evaporate it into air, and spray the air to the outside world, so as to achieve an aromatherapy effect. However, due to the fixed arrangement of the oil storage cavity, when a user adds an essential oil mixture with another scent, the scents may be mixed, so that the aromatherapy effect is poor.

In order to solve the problems of the traditional essential oil diffuser, there is an essential oil diffuser with an external essential oil bottle on the market. For example, the utility model No. 202123047389. X provides an essential oil diffuser, which is mainly composed of an atomizer and a detachable essential oil bottle. There may be a plurality of essential oil bottles to store essential oil mixtures with different scents separately. During use, a corresponding essential oil bottle is connected to the atomizer. Although this essential oil diffuser can improve the aromatherapy effect, there are still the following problems in actual use:

Firstly, during use, the entire essential oil diffuser needs to be placed upside down to drive the essential oil mixture in the essential oil bottle to continuously enter the atomizer under the gravity. This design would cause an excessive burden on the atomizer and shorten the service life of the atomizer. In addition, the essential oil mixture easily spills out of the atomizer during use, resulting in a possibility of oil leakage.

Secondly, during mounting and removal of the essential oil bottle, the essential oil mixture in the essential oil bottle easily spills out, resulting in poor experience.

### SUMMARY

### (I) Technical Problem to be Solved

The present disclosure provides an oil-leakage prevention atomizer and an essential oil diffuser, which can solve the problems mentioned in the background section.

### (I) Technical Solutions

In order to achieve the above objective, the present disclosure provides the following technical solutions:

An oil-leakage prevention atomizer includes a carrier, a reaction chamber, an atomizing core, a separator, and an oil supply chamber, wherein the carrier is provided with a carrier cavity, and an air inlet and an air outlet which are communicated to an outside world are arranged on a cavity wall of the carrier cavity; the reaction chamber is mounted in the carrier cavity, and a reaction cavity is arranged inside the reaction chamber; an oil inlet hole, an atomizing port, and an air hole which are communicated to the carrier cavity are arranged on a cavity wall of the reaction cavity; the atomizing core is mounted at the atomizing port and isolates the reaction cavity from the carrier cavity; the separator is mounted above the reaction cavity and is provided with an oil guide pipe communicated to the oil inlet hole and an air guide pipe communicated to the air hole; and the oil supply chamber sleeves the separator and is connected to the carrier; the separator separates an interior of the oil supply chamber to form an oil storage cavity configured to store an essential oil mixture; a pipe orifice of the oil guide pipe is located at a bottom of the oil storage cavity; the air guide pipe is designed to extend upwards; and a pipe orifice of the air guide pipe extends out of a liquid level of the essential oil mixture.

Preferably, the oil-leakage prevention atomizer further includes an essential oil bottle, wherein a vertical mounting hole and oil falling hole are respectively arranged on a top cavity wall and a bottom cavity wall of the oil storage cavity; an oil guide cavity communicated to the oil falling hole is arranged in the separator; a pipe orifice of the oil guide pipe and the pipe orifice of the air guide pipe are both located in the oil guide cavity, and the pipe orifice of the oil guide pipe is located below the pipe orifice of the air guide pipe; a rubber plug is mounted at a bottle opening of the essential oil bottle; the rubber plug is provided with an oil dripping hole and an air pressure pipe; a first end of the air pressure pipe penetrates through the rubber plug; a second end of the air pressure pipe extends into the essential oil bottle and extends out of the liquid level of the essential oil mixture; the bottle opening of the essential oil bottle can penetrate through the mounting hole into the oil storage cavity and abuts against the oil falling hole, so that the essential oil mixture in the essential oil bottle enters the oil guide cavity via the oil dripping hole and the oil falling hole.

Preferably, a bottom cavity wall of the oil guide cavity gradually tilts from the pipe orifice of the air guide pipe in a direction of the pipe orifice of the oil guide pipe, so as to guide the essential oil mixture in the oil guide cavity to enter the air guide pipe.

Preferably, the carrier cavity extends upwards and surrounds the separator and the oil supply chamber.

Preferably, the atomizing core is an atomizing column; the atomizing column is internally provided with a flue having two end openings and communicated to the carrier cavity; an oil guide hole with oil guide cloth inside is arranged on a side wall of the flue; the atomizing column is mounted at the atomizing port and isolates the reaction cavity from the carrier cavity; the oil guide cloth at the oil guide hole is in contact with the essential oil mixture in the reaction cavity; and the flue is communicated to the air inlet and the air outlet.

Preferably, oil absorption cotton is further mounted in the reaction cavity; a bottom side of the oil absorption cotton is in contact with a bottom cavity wall of the reaction cavity, while a top side of the oil absorption cotton covers the oil guide hole and is in contact with the oil guide cloth to transport the essential oil mixture in the reaction cavity to the oil guide cloth.

An essential oil diffuser includes a machine body, an air supply mechanism, and the above oil-leakage prevention atomizer, wherein the oil-leakage prevention atomizer is detachably mounted on the machine body; the air supply mechanism is mounted inside the machine body and is designed to correspond to the air inlet of the carrier; and the air supply mechanism transports air to the carrier cavity, so that fragrance mist in the carrier cavity is discharged to the outside world via the air outlet.

Preferably, the air supply mechanism is a fan; a placement cavity and a mist cavity which are communicated to each other are arranged inside the machine body; a through hole communicated to the outside world is arranged on a side cavity wall of the placement cavity; a mist discharging port communicated to the outside world is arranged on a side cavity wall of the mist cavity; the fan is mounted at a junction between the mist cavity and the placement cavity; a clamping cavity with an upward opening is further arranged on the machine body; and the oil-leakage prevention atomizer is detachably mounted in the clamping cavity, so that the air inlet and the air outlet are respectively communicated to the fan and the mist cavity to form a complete air flowing channel.

Preferably, the essential oil diffuser further includes a circuit board, wherein the circuit board is mounted in the placement cavity; a bottom cavity wall of the clamping cavity is provided with a pin electrically connected to the circuit board; a connecting block electrically connected to the atomizing core is mounted at a bottom end of the carrier; and when the oil-leakage prevention atomizer is mounted in the clamping cavity, the pin abuts against the connecting block to supply power.

Preferably, the bottom cavity wall of the clamping cavity is further provided with a magnetic suction block; the connecting block is made of a magnetic suction material; and when the oil-leakage prevention atomizer is mounted in the clamping cavity, the connecting block correspondingly attracts the magnetic suction block.

### (III) Beneficial Effects

The present disclosure provides an oil-leakage prevention atomizer and an essential oil diffuser. The oil-leakage prevention atomizer is designed with the carrier to mount the reaction chamber, the atomizing core, the separator, and the oil supply chamber. All the components are assembled for molding, which is convenient for separate production and machining, while ensuring the overall integration degree. The oil-leakage prevention atomizer also at least has the following functions:

Firstly, the oil supply chamber is communicated to the reaction chamber through the oil guide pipe and the air guide pipe, and the atomizing core blocks the atomizing port on the reaction chamber, so that both the oil storage cavity and the reaction cavity are in a closed state, which avoids the essential oil mixture from spilling out and solves the problem of oil leakage and the problem of oil spilling in mounting and removing processes. This not only facilitates transferring and transportation, but also improves the experience effect.

Secondly, as the pipe orifice of the oil guide pipe is located at the bottom of the oil storage cavity, it can be ensured that the essential oil mixture in the oil storage cavity has a trend to completely flow into the oil guide pipe. The design of the air guide pipe is used to maintain an air pressure, which can provide a necessary condition for the essential oil mixture in the oil storage cavity to enter the reaction cavity, thus ensuring that the essential oil mixture can smoothly flow into the reaction cavity through the oil guide pipe, reducing residues of the essential oil mixture in the oil storage cavity, and improving the utilization rate of the essential oil mixture.

Thirdly, as the atomizing core is arranged at a top of the reaction cavity, the gravity of the essential oil mixture entering the reaction cavity will not be applied to the atomizing core during actual use. Moreover, under the action of the air guide pipe, a pressure applied by the essential oil mixture on the atomizing core can be reduced, a load on the atomizing core can be reduced, the service life of the atomizing core can be prolonged, and the possibility of spilling out of the essential oil mixture can be further decreased.

Due to the unique design of the oil-leakage prevention atomizer, the essential oil diffuser with the oil-leakage prevention atomizer will not leak oil during actual use, and will not cause the essential oil mixture to spill out during replacement and mounting, thus improving the user experience. As the machine body and the oil-leakage prevention atomizer are separately designed, when the atomizing core or another part inside the oil-leakage prevention atomizer is damaged, a user can directly replace the damaged oil-leakage prevention atomizer with a new oil-leakage prevention atomizer, instead of replacing the machine body, which effectively reduces the maintenance cost.

### BRIEF DESCRIPTION OF THE DRAWINGS

The accompanying drawings are intended to provide a further understanding of the present disclosure and constitute a part of this specification. The accompanying drawings and embodiments of the present disclosure are used together to explain the present disclosure rather than constituting a limitation on the present disclosure. In the drawings:

FIG. 1 shows a schematic structural diagram I of three kinds of oil-leakage prevention atomizers according to the present disclosure;

FIG. 2 shows a top view of FIG. 1;

FIG. 3 shows a sectional view of A-A of FIG. 2;

FIG. 4 shows a schematic structural diagram II of three kinds of oil-leakage prevention atomizers according to the present disclosure;

FIG. 5 shows a schematic exploded diagram of a first kind of oil-leakage prevention atomizer according to the present disclosure;

FIG. 6 shows a schematic exploded diagram of a second kind of oil-leakage prevention atomizer according to the present disclosure;

FIG. 7 shows a partially schematic structural diagram of a second kind of oil-leakage prevention atomizer according to the present disclosure;

FIG. 8 shows a schematic exploded diagram of a third kind of oil-leakage prevention atomizer according to the present disclosure;

FIG. 9 shows a schematic diagram of usage states of three kinds of oil-leakage prevention atomizers according to the present disclosure;

FIG. 10 shows a schematic diagram of a usage state of a first kind of oil-leakage prevention atomizer according to the present disclosure;

FIG. 11 shows a sectional view of B-B of FIG. 10;

FIG. 12 shows an enlarged view of the part Ain FIG. 11;

FIG. 13 shows a schematic diagram of usage states of second and third kinds of oil-leakage prevention atomizers according to the present disclosure;

FIG. 14 shows a sectional view of C-C of FIG. 13;

FIG. 15 shows an enlarged view of the part B in FIG. 14;

FIG. 16 shows an enlarged view of the part C in FIG. 14;

FIG. 17 shows a schematic exploded diagram of a machine body according to the present disclosure;

FIG. 18 shows a partially schematic structural diagram I of a first kind of machine body according to the present disclosure; and

FIG. 19 shows a partially schematic structural diagram I of a second kind of machine body according to the present disclosure.

In the drawings: 1: carrier; 10: carrier cavity; 101: air inlet; 102: air outlet; 11: connecting block; 2: reaction chamber; 20: reaction cavity; 200: atomizing port; 201: oil inlet hole; 202: air hole; 21: oil absorption cotton; 3: atomizing core; 31: atomizing column; 310: flue; 32: oil guide hole; 320: oil guide cloth; 4: separator; 40: oil guide cavity; 41: oil guide pipe; 42: air guide pipe; 43: stud; 44: separation block; 440: sealing pad; 5: oil supply chamber; 50: oil storage cavity; 501: small hole; 502: chamber cover; 51: mounting hole; 52: oil falling hole; 6: essential oil bottle; 60: rubber plug; 61: oil dripping hole; 62: air pressure pipe; 7: machine body; 71: placement cavity; 710: through hole; 72: mist cavity; 720: mist discharging port; 73: clamping cavity; 731: magnetic suction block; 74: gasket; 8: air supply mechanism; 80: fan; 9: circuit board; 91: pin; 92: control button; and 93: charging port.

### DETAILED DESCRIPTION OF THE EMBODIMENTS

The technical solutions in the embodiments of present disclosure are clearly and completely described below with reference to the accompanying drawings in the embodiments of present disclosure. Apparently, the described embodiments are merely some rather than all of the embodiments of present disclosure. All other embodiments obtained by a person of ordinary skill in the art based on the embodiments of present disclosure without making creative efforts shall fall within the protection scope of present disclosure.

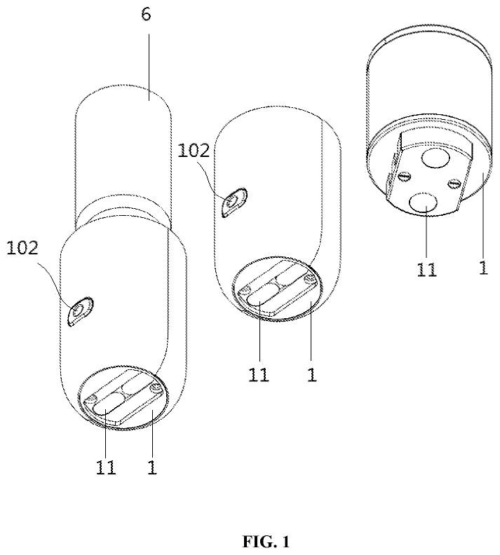

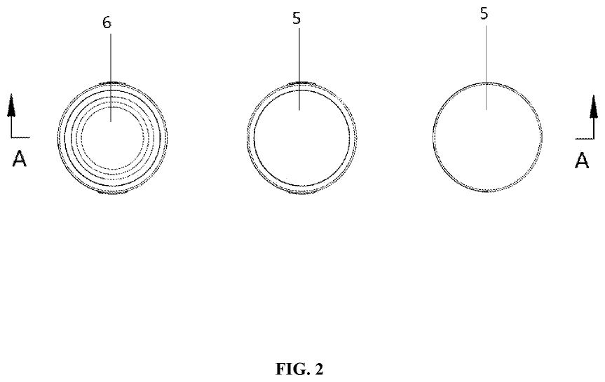

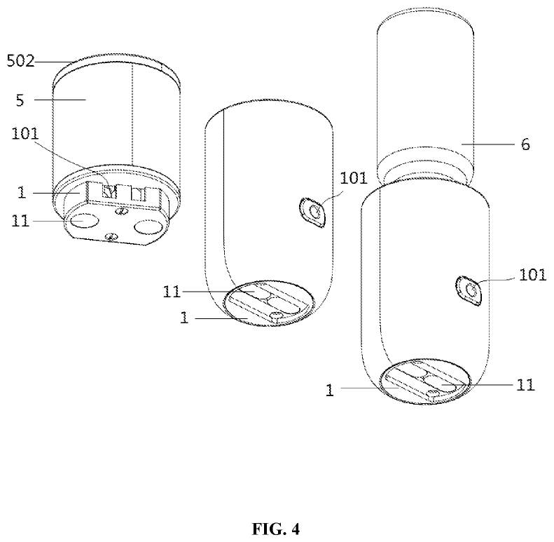

Referring to FIG. 1 to FIG. 6, an oil-leakage prevention atomizer includes a carrier 1, a reaction chamber 2, an atomizing core 3, a separator 4, and an oil supply chamber 5. The carrier 1 is provided with a carrier cavity 10, and an air inlet 101 and an air outlet 102 which are communicated to an outside world are arranged on a cavity wall of the carrier cavity 10. The reaction chamber 2 is mounted in the carrier cavity 10, and a reaction cavity 20 is arranged inside the reaction chamber 2. An oil inlet hole 201, an atomizing port 200, and an air hole 202 which are communicated to the carrier cavity 10 are arranged on a cavity wall of the reaction cavity 20. The atomizing core 3 is mounted at the atomizing port 200 and isolates the reaction cavity 20 from the carrier cavity 10. The separator 4 is mounted above the reaction cavity 2, and the separator 4 is provided with an oil guide pipe 41 communicated to the oil inlet hole 201 and an air guide pipe 42 communicated to the air hole 202. The oil supply chamber 5 sleeves the separator 4 and is connected to the carrier 1. The separator 4 separates an interior of the oil supply chamber 5 to form an oil storage cavity 50 configured to store an essential oil mixture; a pipe orifice of the oil guide pipe 41 is located at a bottom of the oil storage cavity 50. The air guide pipe 42 is designed to extend upwards, and a pipe orifice of the air guide pipe 42 extends out of a liquid level of the essential oil mixture.

Specifically, during production, a certain amount of air needs to be reserved in the oil storage cavity 50 and the reaction cavity 20 to avoid the oil storage cavity 50 and the reaction cavity 20 from being in a vacuum state. In a process of injecting the essential oil mixture into the oil storage cavity 50, under the action of a weight and an air pressure, the essential oil mixture may flow into the reaction cavity 20 along the oil guide pipe and be in contact with the atomizing core 3. After the reaction cavity 20 is filled with the essential oil mixture, and a liquid level difference is formed between the air guide pipe and the oil guide pipe.

When the atomizing core 3 is powered on to work, the essential oil mixture in contact with the atomizing core 3 is atomized into fragrance mist. In this process, when the amount of the essential oil mixture in the reaction cavity 20 gradually decreases, the air guide pipe maintains a balance between an air pressure in the reaction cavity 20 and an air pressure in the oil storage cavity 50, so that the essential oil mixture in the oil storage cavity 50 flows synchronously into the reaction cavity 20 along the oil guide pipe for replenishment, which ensures that the atomizing core 3 can continuously come into contact with the essential oil mixture, thereby ensuring smooth atomization. After the generated fragrance mist enters the carrier cavity 10, external equipment is used to transport an air flow to the air inlet 101, and the fragrance mist is discharged to the outside world from the air outlet 102 to achieve an aromatherapy effect.

In summary, the present disclosure is designed with the carrier 1 to mount the reaction chamber 2, the atomizing core 3, the separator 4, and the oil supply chamber 5. All the components are assembled for molding, which is convenient for separate production and machining, while ensuring the overall integration degree. The oil-leakage prevention atomizer also at least has the following functions:

Firstly, the oil supply chamber 5 is communicated to the reaction chamber 2 through the oil guide pipe and the air guide pipe, and the atomizing core 3 blocks the atomizing port 200 on the reaction chamber 2, so that both the oil storage cavity 50 and the reaction cavity 20 are in a closed state, which avoids the essential oil mixture from spilling out and solves the problem of oil leakage and the problem of oil spilling in mounting and removing processes. This not only facilitates transferring and transportation, but also improves the experience effect.

Secondly, as the pipe orifice of the oil guide pipe is located at the bottom of the oil storage cavity 50, it can be ensured that the essential oil mixture in the oil storage cavity 50 has a trend to completely flow into the oil guide pipe. The design of the air guide pipe is used to maintain an air pressure, which can provide a necessary condition for the essential oil mixture in the oil storage cavity 50 to enter the reaction cavity 20, thus ensuring that the essential oil mixture can smoothly flow into the reaction cavity 20 through the oil guide pipe, reducing residues of the essential oil mixture in the oil storage cavity 50, and improving the utilization rate of the essential oil mixture.

Thirdly, as the atomizing core 3 is arranged at a top of the reaction cavity 20, the gravity of the essential oil mixture entering the reaction cavity 20 will not be applied to the atomizing core 3 during actual use. Moreover, under the action of the air guide pipe, a pressure applied by the essential oil mixture on the atomizing core 3 can be reduced, a load on the atomizing core 3 can be reduced, the service life of the atomizing core 3 can be prolonged, and the possibility of spilling out of the essential oil mixture can be further decreased.

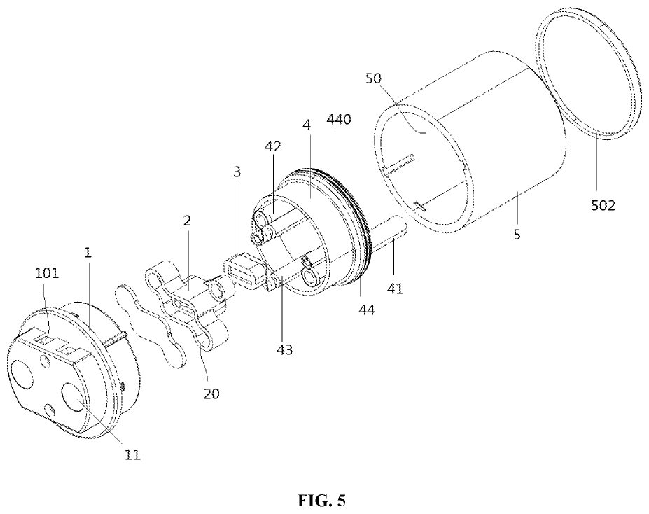

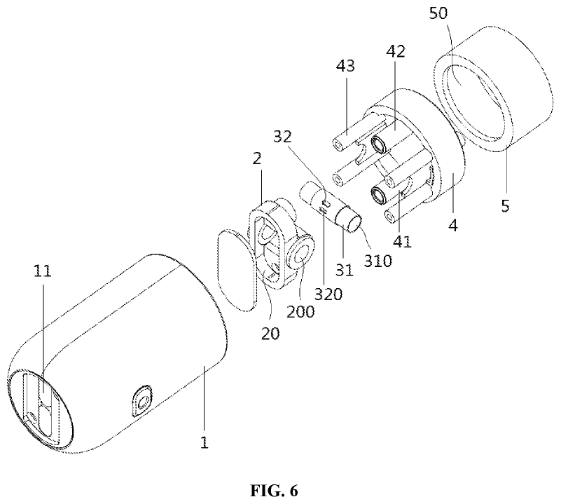

It should be noted that the above content only limits the oil-leakage prevention atomizers in FIG. 5 and FIG. 6. The oil-leakage prevention atomizer in FIG. 5 is clearly recorded in the preceding application documents, and the oil-leakage prevention atomizer in FIG. 6 does not exceed the scope recorded in the preceding application documents, thus enjoying the priority.

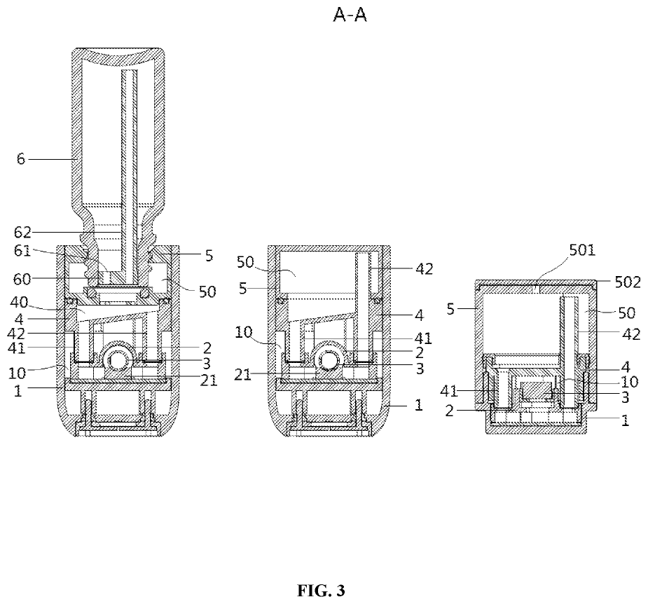

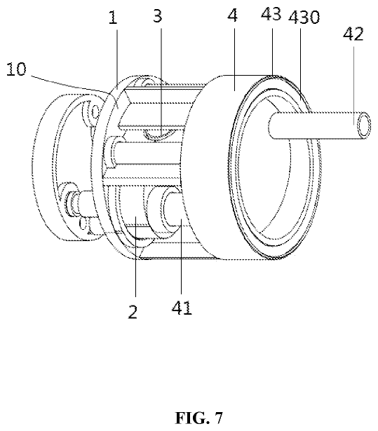

Referring to FIG. 3 to FIG. 8, in order to facilitate production and machining of parts and make the oil-leakage prevention atomizer easier to assemble, remove, and maintain, in the present disclosure, the separator 4 is provided with studs 43, and the carrier 1 is fixed to the studs 43 through screws, so that the separator 4 is fixed to the carrier 1, and the oil guide pipe 41 and the air guide pipe 42 on the separator 4 press against and limit the reaction chamber 2 on the carrier 1. As there is no related electrical part inside the oil supply chamber 5, the oil supply chamber can also be directly adhered or buckled on the carrier 1.

Referring to FIG. 5 to FIG. 8, in order to improve the leakproofness, the separator 4 is further provided with a separation block 44, and a sealing pad 440 resisting against a side wall of the oil storage cavity 50 is mounted on the separation block 44. Sealing rings are mounted at a connection between the oil guide pipe 41 and the oil inlet hole 201 and a connection between the air guide pipe 42 and the air hole 202.

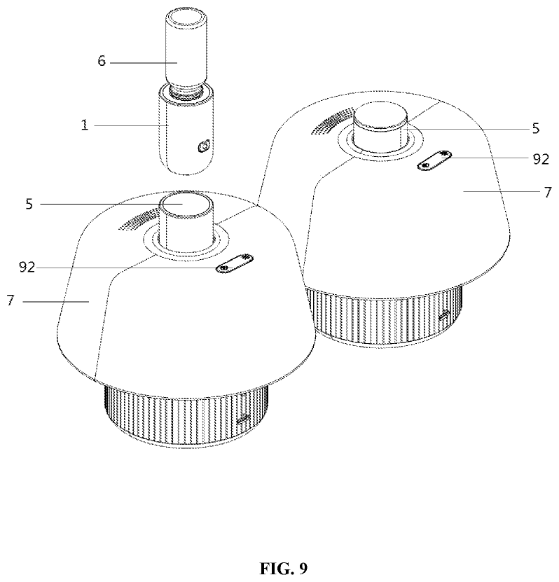

Referring to FIG. 5 and FIG. 9, there are various ways to add the essential oil mixture into the oil storage cavity 50, specifically as follows:

Pre-filling: In an assembling process, the oil supply chamber 5 is directly placed upside down; and after a fixed volume of an essential oil mixture is placed in the oil storage cavity 50, the connected and formed carrier 1, reaction chamber 2, and separator 4 are assembled with the upside-down oil supply chamber 5.

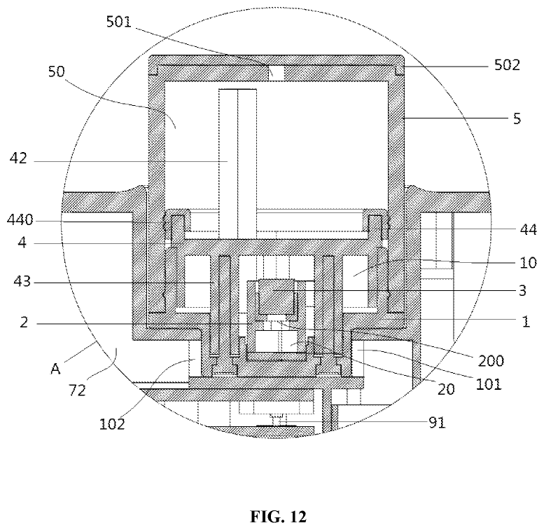

Later filling: As shown in FIG. 12, a small hole 501 communicated to the oil storage cavity 50 is designed on the oil supply chamber 5. An injector is used to inject the essential oil mixture from the small hole 501 into the oil storage cavity 50, and then the small hole 501 is blocked through a chamber cover 502.

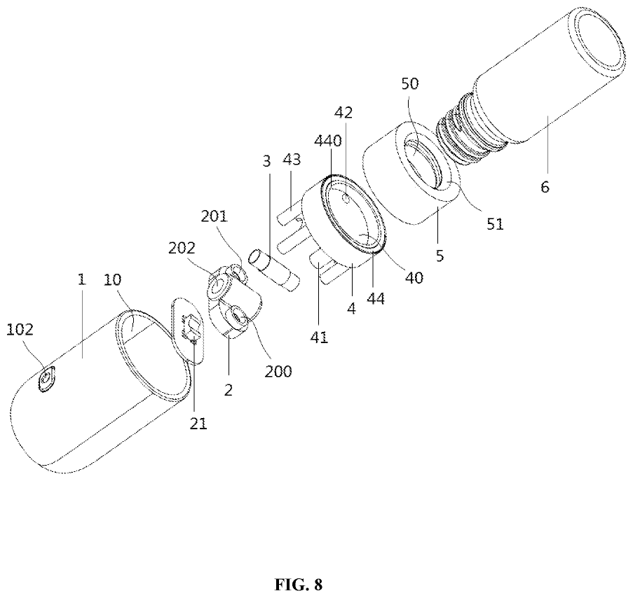

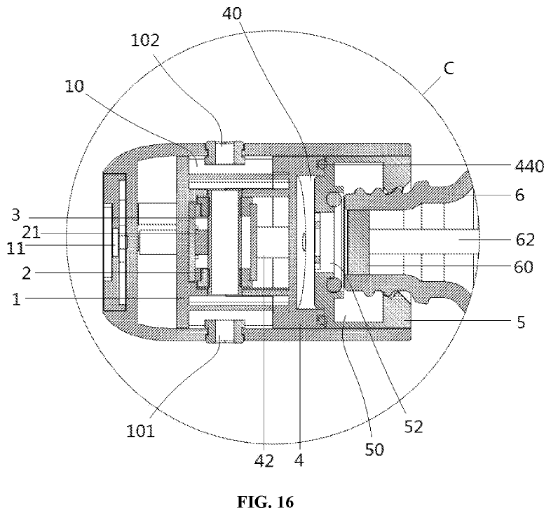

Referring to FIG. 1, FIG. 3, FIG. 8, and FIG. 16, it is found during actual use that if the essential oil is filled using the pre-filling way, the entire oil-leakage prevention atomizer needs to be replaced when the essential oil mixture in oil supply chamber 5 is used up. Although the small hole 501 arranged on the oil supply chamber 5 can be used for multiple times, due to a small hole position, it is not convenient for a user to perform an oil filling operation. To solve this problem, the oil-leakage prevention atomizer further includes an essential oil bottle 6. A vertical mounting hole 51 and oil falling hole 52 are respectively arranged on a top cavity wall and a bottom cavity wall of the oil storage cavity 50. An oil guide cavity 40 communicated to the oil falling hole 52 is arranged in the separator 4. A pipe orifice of the oil guide pipe 41 and the pipe orifice of the air guide pipe 42 are both located in the oil guide cavity 40, and the pipe orifice of the oil guide pipe 41 is located below the pipe orifice of the air guide pipe 42. A rubber plug 60 is mounted at a bottle opening of the essential oil bottle 6. The rubber plug 60 is provided with an oil dripping hole 61 and an air pressure pipe 62. A first end of the air pressure pipe 62 penetrates through the rubber plug 60. A second end of the air pressure pipe 62 extends into the essential oil bottle 6 and extends out of the liquid level of the essential oil mixture. The bottle opening of the essential oil bottle 6 can penetrate through the mounting hole 51 into the oil storage cavity 50 and abuts against the oil falling hole 52, so that the essential oil mixture in the essential oil bottle 6 enters the oil guide cavity 40 via the oil dripping hole and the oil falling hole 52.

Specifically, a diameter of the oil dripping hole 61 is small, so that it is hard for the essential oil mixture to spill out through the oil dripping hole 61 without being compressed by an air pressure. In a process of mounting the essential oil bottle 6, since the second end of the air pressure pipe 62 is arranged in the essential oil mixture, a probability of spilling out of the essential oil mixture can be effectively reduced. After the mounting of the essential oil bottle 6 is completed, the bottle opening of the essential oil bottle 6 may penetrate through the mounting hole 51 and abut against the oil dripping hole 52, so that the essential oil bottle 6 is completely vertical and is communicated to the oil guide cavity 40. At this time, the second end of the air pressure pipe 62 extends out of the liquid level of the essential oil mixture, so that an air pressure difference is generated between the inside and outside of the essential oil bottle 6, thereby pressing the essential oil mixture out of the oil dripping hole 61 and dripping the essential oil mixture into the oil guide cavity 40. Therefore, the cooperation design of the air pressure pipe 62 and the oil dripping hole 61 can reduce the spilling amount of the essential oil mixture and improve the user experience.

The essential oil mixture that initially enters the oil guide cavity 40 may flow under the action of the gravity towards the oil guide pipe 41 below and enter the reaction cavity 20 through the oil guide pipe 41. In this process, the essential oil mixture will compress the air in the reaction cavity 20, and the compressed air is discharged into the oil guide cavity 40 through the air hole 202 and the air guide pipe 42. Therefore, the cooperation design of the air hole 202 and the air guide pipe 42 at this time can discharge the air in the reaction cavity 20 and provide conditions for the essential oil mixture to enter the reaction cavity 20.

As the essential oil mixture in the essential oil bottle 6 continues to be dripped, the oil guide cavity 40, the oil guide pipe 41, the reaction cavity 20, and the air guide pipe 42 are gradually filled with the essential oil mixture. In this process, the air pressed out of the reaction cavity 20 may be moved up to an empty region at a top of the essential oil bottle 6 through the air pressure pipe 62 under the pressing effect of the essential oil mixture. After the oil guide cavity 40 is completely filled, the essential oil mixture in the essential oil bottle 6 is mixed with the essential oil mixture in the oil guide cavity 40 and maintains a balanced state. In this state, the essential oil bottle 6 may continue to drip the essential oil mixture into the oil guide cavity 40 for replenishment only when the atomizing core 3 heats and volatilizes the essential oil mixture in the reaction cavity 20.

Referring to FIG. 8 and FIG. 16, the bottle opening of the essential oil bottle 6 is in threaded connection with the mounting hole 51 to facilitate the user to mount and remove the essential oil bottle 6.

Referring to FIG. 3, FIG. 8, and FIG. 16, a bottom cavity wall of the oil guide cavity 40 gradually tilts from the pipe orifice of the air guide pipe 42 in a direction of the pipe orifice of the oil guide pipe 41. This design can cause the essential oil mixture falling into the oil guide cavity 40 to enter the air guide pipe 42 more easily.

It should be noted that the above content is used to limit the oil-leakage prevention atomizer in FIG. 8, and this design is an improvement based on the solutions recorded in the preceding application documents.

Referring to FIG. 1 to FIG. 8, the carrier cavity 10 extends upwards and surrounds the separator 4 and the oil supply chamber 5. On the one hand, this design can ensure that the carrier 1 can stably limit the separator 4 and the oil supply chamber 5 to avoid separation; and on the other hand, the leakproofness between the separator 4 and the oil supply chamber 5 can also be improved, and the influence on use caused by the seepage of the essential oil mixture to the outside world can be avoided.

Referring to FIG. 1 to FIG. 9, the atomizing core 3 belongs to an existing product in various types. For example, a planar ceramic atomizing core 3 as shown in FIG. 5 can be used, or an atomizing column 31 as shown in FIG. 6 and FIG. 8 can be used. In order to improve the use experience, in the present disclosure, the atomizing core 3 is the atomizing column 31. The atomizing column 31 is internally provided with a flue 310 having two end openings and communicated to the carrier cavity 10. An oil guide hole 32 with oil guide cloth 320 inside is arranged on a side wall of the flue 310. The atomizing column 31 is mounted at the atomizing port 200 and isolates the reaction cavity 20 from the carrier cavity 10. The oil guide cloth 320 at the oil guide hole 32 is in contact with the essential oil mixture in the reaction cavity 20. The flue 310 is communicated to the air inlet 101 and the air outlet 102.

Specifically, after the essential oil mixture entering the reaction cavity 20 is accumulated to a certain height, the essential oil mixture may be in contact with the oil guide cloth 320 in the oil guide hole 32. At this time, the atomizing column 31 is powered on to heat and volatilize the essential oil mixture in the oil guide cloth 320. The generated fragrance mist flows into the flue 310 and the carrier cavity 10, and an air flow is transported to the air inlet 101 using the external equipment. The fragrance mist is discharged from the air outlet 102 to the outside world to achieve the aromatherapy effect.

Compared to the conventional atomizing core 3, the atomizing column 31 is designed with the oil guide cloth 320 to enlarge a contact area with the essential oil mixture, so that the volatilization efficiency is improved. The design of the flue 310 can achieve concentrated discharging of the fragrance mist, so as to reduce the amount of the fragrance mist remaining in the carrier cavity 10, thus effectively improving the usage effect.

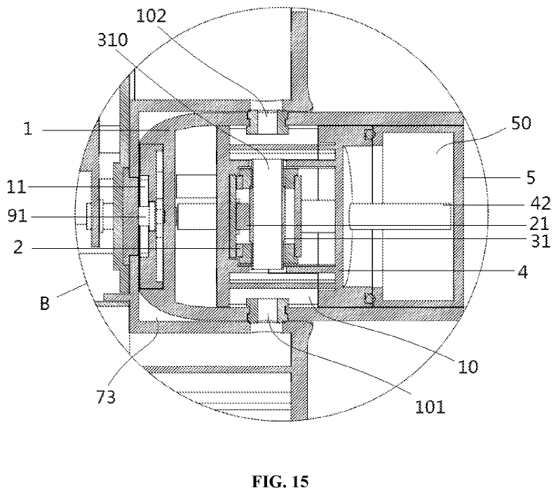

Referring to FIG. 6 to FIG. 8 and FIG. 14 to FIG. 16, when the liquid level of the essential oil mixture in the reaction cavity 20 is low, there is residual essential oil mixture due to a failure of being in contact the oil guide cloth 320, and the residual essential oil mixture may be mixed with an essential oil mixture with another scent to produce smoke with a peculiar smell. When the reaction cavity 20 is in a fully filled state, the essential oil mixture easily passes through the oil guide cloth 320 and seeps into the flue 310, which affects the normal use. To solve the above problems, in the present disclosure, oil absorption cotton 21 is further mounted in the reaction cavity 20. A bottom side of the oil absorption cotton 21 is in contact with a bottom cavity wall of the reaction cavity 20, while a top side of the oil absorption cotton 21 covers the oil guide hole 32 and is in contact with the oil guide cloth 320 to transport the essential oil mixture in the reaction cavity 20 to the oil guide cloth 320.

Specifically, during use, the oil absorption cotton 21 can absorb the residual essential oil mixture in the reaction cavity 20 and transport the residual essential oil mixture to the oil guide cloth 320, so that the residual essential oil mixture is volatilized normally. When the reaction cavity 20 is full of the essential oil mixture, the essential oil mixture may enter the oil absorption cotton 21 and fill a large number of small pores of the oil absorption cotton, so that the entire oil absorption cotton 21 is in a closed and saturated state. At this time, the oil absorption cotton loses its absorption capacity and can resist continued seepage of the remaining essential oil mixture by a surface tension of the essential oil mixture, thus avoiding the essential oil mixture in the reaction cavity 20 from being in contact with the oil guide cloth 320 when the atomizing column 31 does not work.

When the atomizing column 31 works, generated heat heats and volatilizes the essential oil mixture of the oil guide cloth 320. The dry oil guide cloth 320 then absorbs the essential oil mixture from the oil absorption cotton 21, causing the oil absorption cotton 21 to exit the saturated state. The oil absorption cotton recovers its absorption capacity and can continue to absorb the essential oil mixture from the reaction cavity 20 to maintain the saturated state. This operation is performed repeatedly until the essential oil in the reaction cavity 20 is completely absorbed to the oil guide cloth 320 for being heated.

Therefore, on the one hand, the design of the oil absorption cotton 21 can effectively avoid the essential oil mixture in the reaction cavity 20 from spilling into the flue 310, so that the problem of oil leakage is solved. On the other hand, this design can prevent the residual essential oil mixture in the reaction cavity 20, which can not only avoid a waste, but also solve the problem of peculiar smell.

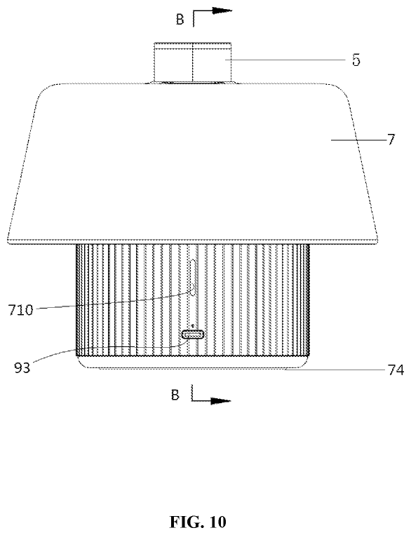

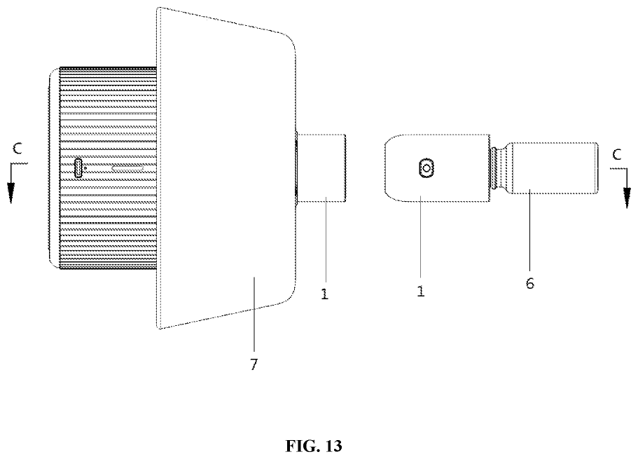

Referring to FIG. 9 to FIG. 19, an essential oil diffuser includes a machine body 7, an air supply mechanism 8, and an oil-leakage prevention atomizer. The oil-leakage prevention atomizer is detachably mounted on the machine body 7. The air supply mechanism 8 is mounted inside the machine body 7 and is designed to correspond to the air inlet 101 of the carrier 1; and the air supply mechanism 8 transports air to the carrier cavity 10 to discharge fragrance mist in the carrier cavity 10 to the outside world from the air outlet 102.

Specifically, during use, the oil-leakage prevention atomizer with an essential oil mixture having a corresponding scent is firstly mounted on the machine body 7, and the atomizing core 3 is activated to generate fragrance mist into the carrier cavity 10. The air supply mechanism 8 is then activated to transport air to the air inlet 101 and the carrier cavity 10, so that the fragrance mist inside the carrier cavity 10 is discharged from the air outlet 102 to the outside world. The oil-leakage prevention atomizer and the machine body 7 are designed to be detachable, so that it is convenient for a user to remove and replace the oil-leakage prevention atomizer.

In summary, due to the unique design of the oil-leakage prevention atomizer, the essential oil diffuser with the oil-leakage prevention atomizer will not leak oil during actual use, and will not cause the essential oil to spill out during replacement and mounting, thus improving the user experience. As the machine body 7 and the oil-leakage prevention atomizer are separately designed, when the atomizing core 3 or another part inside the oil-leakage prevention atomizer is damaged, the user can directly replace the damaged oil-leakage prevention atomizer with a new oil-leakage prevention atomizer, instead of replacing the machine body 7, which effectively reduces the maintenance cost.

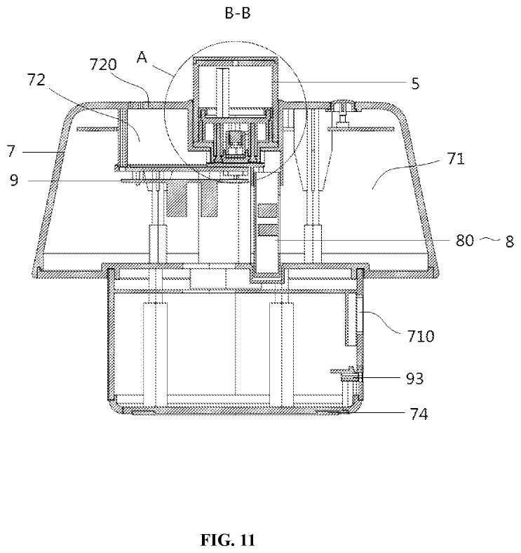

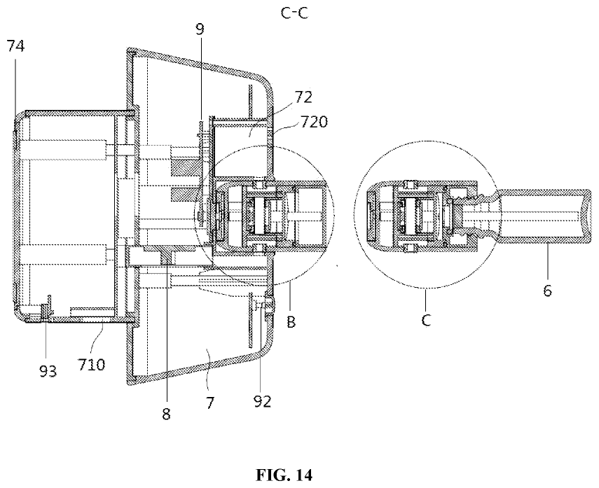

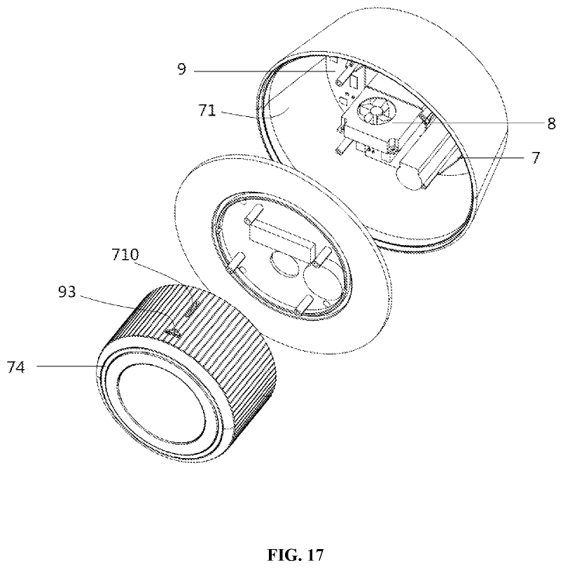

Referring to FIG. 11, FIG. 14, and FIG. 17, further, there are a variety of air supply mechanisms 8. For example, an air pump, an air blower, and the like can be used. In the present disclosure, the air supply mechanism 8 is preferably a fan 80. A placement cavity 71 and a mist cavity 72 which are communicated to each other are arranged inside the machine body 7. A through hole 710 communicated to the outside world is arranged on a side cavity wall of the placement cavity 71. A mist discharging port 720 communicated to the outside world is arranged on a side cavity wall of the mist cavity 72. The fan 80 is mounted at a junction between the mist cavity 72 and the placement cavity 71. A clamping cavity 73 with an upward opening is further arranged on the machine body 7. The oil-leakage prevention atomizer is detachably mounted in the clamping cavity 73, so that the air inlet 101 and the air outlet 102 are respectively communicated to the fan 80 and the mist cavity 72 to form a complete air flowing channel.

Specifically, during use, one end of the oil-leakage prevention atomizer that is provided with the carrier 1 is downwards clamped into the clamping cavity 73, and it is ensured that the air inlet 101 is communicated to the fan 80 and the air outlet 102 is communicated to the mist cavity 72, thus forming the complete air flowing channel. Next, the fan 80 is activated to suck external air from the through hole 710, so that the air passes through the air inlet 101, the carrier cavity 10, the air outlet 102, and the mist cavity 72 in sequence, and is finally discharged to the outside world from the mist discharging port 720. In this process, the atomizing core 3 is powered on at the same time, so that the generated fragrance mist can enter the carrier cavity 10 and be trapped by the air and timely taken to the outside world to achieve an aromatherapy effect.

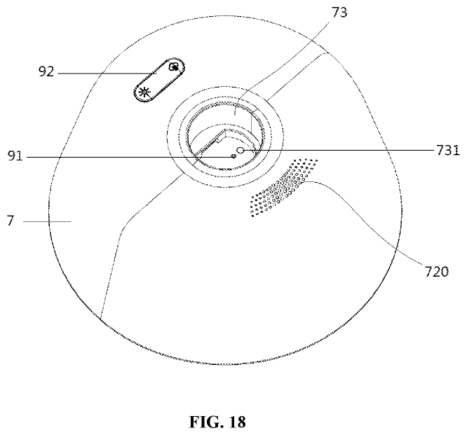

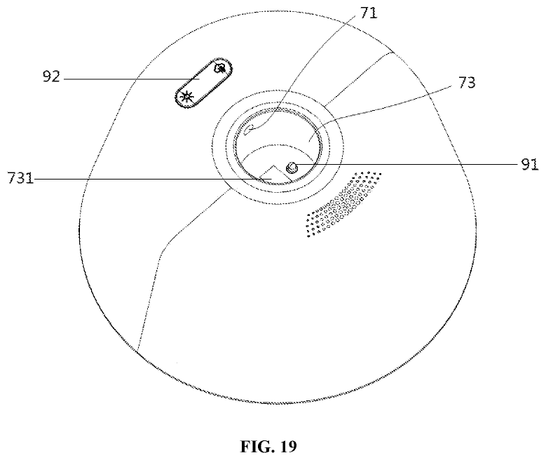

It should be noted that as shown in FIG. 11, FIG. 14, FIG. 18, and FIG. 19, manufacturers can set the corresponding clamping cavity 73 according to a size of the carrier 1 and set a corresponding air path in the clamping cavity 73 according to positions of the air inlet 101 and the air outlet 102, so that the machine body 7 of the essential oil diffuser can adapt to different types of oil-leakage prevention atomizers.

Referring to FIG. 11, FIG. 14, and FIG. 17, regarding a power supplying method for the atomizing core 3, a storage battery can be directly arranged in the carrier cavity 10 to supply power, or an external power source can be directly used to supply power. The present disclosure does not make limitations on this. For the convenience of understanding, the essential oil diffuser further includes a circuit board 9. The circuit board 9 is mounted in the placement cavity 71. A bottom cavity wall of the clamping cavity 73 is provided with a pin 91 electrically connected to the circuit board 9. A connecting block 11 electrically connected to the atomizing core 3 is mounted on the carrier 1. When the oil-leakage prevention atomizer is mounted in the clamping cavity 73, the connecting block 11 abuts against the pin 91.

Specifically, when the connecting block 11 corresponds to the pin 91, power can be directly supplied to the pin 91 through the circuit board 9, so that a current is made to the connecting block 11 to activate the atomizing core 3. When it is necessary to turn off an atomizing function, the circuit board 9 is controlled to stop supplying power to the pin 91. Similarly, to replace the oil-leakage prevention atomizer, it is necessary to first control the circuit board 9 to stop supplying power to the atomizing core 3, then lift the oil-leakage prevention atomizer to separate the connecting block 11 from the pin 91, and remove the oil-leakage prevention atomizer from the machine body 7.

Referring to FIG. 18 to FIG. 19, the bottom cavity wall of the clamping cavity 73 is further provided with a magnetic suction block 731. The connecting block 11 is made of a magnetic suction material. When the oil-leakage prevention atomizer is mounted in the clamping cavity 73, the connecting block 11 correspondingly attracts the magnetic suction block 731.

Specifically, when the oil-leakage prevention atomizer is mounted in the clamping cavity 73, the connecting block 11 correspondingly attracts the magnetic suction block 731, which can fix the oil-leakage prevention atomizer on the machine body 7. Similarly, when the user upwards pulls the oil-leakage prevention atomizer with a certain force, the connecting block 11 can be separated from the magnetic suction block 731 to remove the oil-leakage prevention atomizer from the machine body 7. Therefore, the magnetic suction method used for connecting the machine body 7 to the oil-leakage prevention atomizer not only facilitates production, machining, and mounting, but also improves the handfeel of operation of the user.

It should be noted that the connecting block 11 can be made of a magnetic and conductive material. For example, an outer layer of an iron material can be plated with gold. Iron is magnetic, and the gold-plated layer is conductive. When the connecting block 11 is electrically connected to the atomizing core 3 through a wire, a current can be transmitted to the atomizing core 3. The iron can also be replaced with cobalt, nickel, or the like, and the gold can also be replaced with copper, silver, or the like. The present disclosure does not make limitations on a specific combination.

Referring to FIG. 9, FIG. 18, and FIG. 19, a control button 92 electrically connected to the circuit board 9 is also mounted on the machine body 7. In a production process, a preset control program needs to be input to the circuit board 9. The user can activate the relevant program on the circuit board 9 by pressing the corresponding control button 92, so that LEDs on a lamp panel are extinguished and lit up, and the circuit board 9 supplies power and stop supplying power to the atomizing core 3. Since controlling electrical elements through the circuit board 9 is the existing technology, a specific circuit structure will not be described in detail in the present disclosure.

Referring to FIG. 9, FIG. 18, and FIG. 19, a charging port 93 electrically connected to the circuit board 9 is further arranged on the machine body 7. The charging port 93 can be connected to an external power source to supply power to the circuit board 9. A storage battery can also be arranged inside the placement cavity 71 to store electrical energy and supply power to the circuit board 9. Since the related technologies are conventional technologies, a specific circuit structure will not be further described in the present disclosure.

Referring to FIG. 11 and FIG. 14, a pad 74 is arranged at a bottom of the machine body 7. This design can increase a friction force on a desktop, which effectively avoids a supporting seat from slipping, improves the placement stability, and facilitates the user to mount and remove the essential oil bottle 6.

It should be also noted that although the embodiments of the present disclosure have been shown and described, it can be understood by those of ordinary skill in the art that various changes, modifications, substitutions, and variations can be made to these embodiments without departing from the principle and spirit of the present disclosure.

What is claimed is:

1. A oil-leakage prevention atomizer, comprising:
a carrier, wherein the carrier is provided with a carrier cavity, and an air inlet and an air outlet which are communicated to an outside world are arranged on a cavity wall of the carrier cavity;
a reaction chamber, wherein the reaction chamber is mounted in the carrier cavity, and a reaction cavity is arranged inside the reaction chamber; an oil inlet hole, an atomizing port, and an air hole which are communicated to the carrier cavity are arranged on a cavity wall of the reaction cavity;
an atomizing core, wherein the atomizing core is mounted at the atomizing port and isolates the reaction cavity from the carrier cavity;
a separator, wherein the separator is mounted above the reaction cavity and is provided with an oil guide pipe communicated to the oil inlet hole and an air guide pipe communicated to the air hole; and
an oil supply chamber, wherein the oil supply chamber sleeves the separator and is connected to the carrier; the separator separates an interior of the oil supply chamber to form an oil storage cavity configured to store an essential oil mixture; a pipe orifice of the oil guide pipe is located at a bottom of the oil storage cavity; the air guide pipe is designed to extend upwards; and a pipe orifice of the air guide pipe extends out of a liquid level of the essential oil mixture.

2. The oil-leakage prevention atomizer according to claim 1, further comprising an essential oil bottle, wherein a vertical mounting hole and oil falling hole are respectively arranged on a top cavity wall and a bottom cavity wall of the oil storage cavity; an oil guide cavity communicated to the oil falling hole is arranged in the separator; a pipe orifice of the oil guide pipe and the pipe orifice of the air guide pipe are both located in the oil guide cavity, and the pipe orifice of the oil guide pipe is located below the pipe orifice of the air guide pipe; a rubber plug is mounted at a bottle opening of the essential oil bottle; the rubber plug is provided with an oil dripping hole and an air pressure pipe; a first end of the air pressure pipe penetrates through the rubber plug; a second end of the air pressure pipe extends into the essential oil bottle and extends out of the liquid level of the essential oil mixture; the bottle opening of the essential oil bottle penetrates through the mounting hole into the oil storage cavity and abuts against the oil falling hole, so that the essential oil mixture in the essential oil bottle enters the oil guide cavity via the oil dripping hole and the oil falling hole.

3. The oil-leakage prevention atomizer according to claim 2, wherein a bottom cavity wall of the oil guide cavity gradually tilts from the pipe orifice of the air guide pipe in a direction of the pipe orifice of the oil guide pipe, so as to guide the essential oil mixture in the oil guide cavity to enter the air guide pipe.

4. The oil-leakage prevention atomizer according to claim 1 or 3, wherein the carrier cavity extends upwards and surrounds the separator and the oil supply chamber.

5. The oil-leakage prevention atomizer according to claim 1 or 3, wherein the atomizing core is an atomizing column; the atomizing column is internally provided with a flue having two end openings and communicated to the carrier cavity; an oil guide hole with oil guide cloth inside is arranged on a side wall of the flue; the atomizing column is mounted at the atomizing port and isolates the reaction cavity from the carrier cavity; the oil guide cloth at the oil guide hole is in contact with the essential oil mixture in the reaction cavity; and the flue is communicated to the air inlet and the air outlet.

6. The oil-leakage prevention atomizer according to claim 1 or 3, wherein oil absorption cotton is further mounted in the reaction cavity; a bottom side of the oil absorption cotton is in contact with a bottom cavity wall of the reaction cavity, while a top side of the oil absorption cotton covers the oil guide hole and is in contact with the oil guide cloth to transport the essential oil mixture in the reaction cavity to the oil guide cloth.

7. An essential oil diffuser, comprising a machine body, an air supply mechanism, and the oil-leakage prevention atomizer according to claim 1 or 3, wherein the oil-leakage prevention atomizer is detachably mounted on the machine body; the air supply mechanism is mounted inside the machine body and is designed to correspond to the air inlet of the carrier; and the air supply mechanism transports air to the carrier cavity to discharge fragrance mist in the carrier cavity to the outside world from the air outlet.

8. The essential oil diffuser according to claim 7, wherein the air supply mechanism is a fan; a placement cavity and a mist cavity which are communicated to each other are arranged inside the machine body; a through hole communicated to the outside world is arranged on a side cavity wall of the placement cavity; a mist discharging port communicated to the outside world is arranged on a side cavity wall of the mist cavity; the fan is mounted at a junction between the mist cavity and the placement cavity; a clamping cavity with an upward opening is further arranged on the machine body; and the oil-leakage prevention atomizer is detachably mounted in the clamping cavity, so that the air inlet and the air outlet are respectively communicated to the fan and the mist cavity to form a complete air flowing channel.

9. The essential oil diffuser according to claim 8, further comprising a circuit board, wherein the circuit board is mounted in the placement cavity; a bottom cavity wall of the clamping cavity is provided with a pin electrically connected to the circuit board; a connecting block electrically connected to the atomizing core is mounted on the carrier; and when the oil-leakage prevention atomizer is mounted in the clamping cavity, the pin abuts against the connecting block to supply power.

10. The essential oil diffuser according to claim 9, wherein the bottom cavity wall of the clamping cavity is further provided with a magnetic suction block; the connecting block is made of a magnetic suction material; and when the oil-leakage prevention atomizer is mounted in the clamping cavity, the connecting block correspondingly attracts the magnetic suction block.
